(19) United States
(12) Patent Application Publication
(10) Pub. No.: US 2014/0024886 A1
(43) Pub. Date: Jan. 23, 2014

(54) BOWEL PACKING DEVICE HAVING A SUPPORT STRUCTURE

(71) Applicants: Anthony P. Deasey, Severna Park, MD (US); Steven J. Kubisen, Ellicott City, MD (US); David J. Hicks, Rockville, MD (US); Dennis Smith, Ellicott City, MD (US)

(72) Inventors: Anthony P. Deasey, Severna Park, MD (US); Steven J. Kubisen, Ellicott City, MD (US); David J. Hicks, Rockville, MD (US); Dennis Smith, Ellicott City, MD (US)

(21) Appl. No.: 14/039,014

(22) Filed: Sep. 27, 2013

Related U.S. Application Data
(63) Continuation of application No. 13/166,635, filed on Jun. 22, 2011, now abandoned.
(60) Provisional application No. 61/392,462, filed on Oct. 12, 2010.

(51) Int. Cl. A61B 17/02

(52) U.S. Cl. CPC A61B 17/0218
USPC 600/37

(57) ABSTRACT

An elastomeric device for packing the bowels of a subject comprising: a central portion and one or more flaps collectively manually positionable within the subject to retain the bowels of the subject in an operational, displaced position and to provide a surgical operational space; and a support structure disposed in at least one of the central portion and the flaps configured to provide rigidity to the device.

### CROSS-REFERENCE TO RELATED APPLICATIONS

This application is a Continuation Application of U.S. patent application Ser. No. 13/166,635 filed Jun. 22, 2011, which claims priority to U.S. Provisional Patent Application No. 61/392,462, filed on Oct. 12, 2010. The contents of U.S. Provisional Patent Application No. 61/392,462 filed on Oct. 12, 2010, U.S. Provisional Patent Application 61/125,219 filed on Apr. 23, 2008, and PCT/US2009/002495 filed on Apr. 22, 2009, are hereby incorporated herein by reference in their entirety.

### BACKGROUND

1. Field of the Invention

The present invention relates generally to bowel packing, and more particularly, to a bowel packing device having a support structure.

2. Related Art

Abdominal and pelvic procedures generally require displacement and retention of bowels or other organs to create a space that allows the surgeon to perform the procedure. This step of displacement and retention of bowels is referred to herein as bowel packing

The current packing procedure used in the operating room today is time-consuming relative to the overall priorities of events in a surgery. The surgeon first uses his hands to displace the bowels away from the surgical site. Intra-abdominal surgical sponges and towels are then used to pack the bowels out of the way. Finally, abdominal retractors are fitted over the dressings with gentle fraction to hold the cotton sponges in place.

This conventional bowel packing causes several issues during surgery. For instance, bowel packing may take up to ten minutes, and, because the bowels have a tendency to protrude from the dressing into the surgical space, the bowel packing must be repeated frequently during extended surgical procedures. Additionally, the cotton sponges used to pack the bowels are made of loose cotton fibers that can adhere to the bowels, and remain within the subject even after removal of the sponges. These fibers can promote peritoneal inflammation, a major cause of post-operative adhesion formation. Furthermore, the sponges tend to dry out over the course of the surgical procedure, becoming abrasive and adhesive to the bowels themselves, further contributing to the formation of adhesions, a leading cause of post-operative morbidity. Finally, because multiple sponges are used, there is a danger that one or more sponges will be forgotten in the abdominal cavity.

### SUMMARY

According to one aspect of the present invention, an elastomeric device for packing the bowels of a subject is disclosed. The device comprises a central portion and one or more flaps collectively manually positionable within the subject to retain the bowels of the subject in an operational, displaced position and to provide a surgical operational space; and a support structure disposed in at least one of the central portion and the flaps configured to provide rigidity to the device.

According to another aspect of the present invention, a device for packing the bowels of a subject is disclosed. The device comprises a central portion and one or more flaps collectively manually positionable within the subject to retain the bowels of the subject in an operational, displaced position and to provide a surgical operational space, wherein at least one of the central portion and the one or more flaps body has a section formed from a thermally-responsive material having a first stiffness when the material is at a first temperature that is above the subject's body temperature, and a second, greater stiffness when the body temperature is approximately at the subject's body temperature.

According to yet another aspect of the present invention, a method of packing bowels of a subject is disclosed. The device includes a central portion and one or more flaps, wherein the device has a section formed from of a thermally-responsive material having a first stiffness when the material is at a first temperature that is above the subject's body temperature, and a second, greater stiffness when the body temperature is approximately at the subject's body temperature, comprising accessing an interior of an abdominal cavity of the subject; repositioning the bowels to provide a surgical space in the abdominal cavity; adding thermal energy to the device to increase the temperature of the device; positioning the device abutting the bowels; and allowing the device to cool to the subject's body temperature such that the device provides a barrier between the bowels and the surgical space.

### BRIEF DESCRIPTION OF THE DRAWINGS

Embodiments of the present invention are described below with reference to the attached drawings, in which:

FIG. 1A is a front view of a bowel packing device having a support structure, in accordance with embodiments of the present invention;

FIG. 1B is a side view of the device of FIG. 1A;

FIG. 1C is a top view of the device of FIG. 1A;

FIG. 1D is a perspective view of the device of FIG. 1A;

FIG. 2A is a front view of a bowel packing device having a support structure, in accordance with embodiments of the present invention;

FIG. 2B is a front view of a bowel packing device having a support structure, in accordance with embodiments of the present invention;

FIG. 2C is a front view of a bowel packing device having a support structure, in accordance with embodiments of the present invention;

FIG. 2D is a front view of a bowel packing device having a support structure, in accordance with embodiments of the present invention;

FIG. 3 is a cross-sectional view of a support structure, in accordance with embodiments of the present invention;

FIG. 4 is a front view of a bowel packing device having a support structure, in accordance with embodiments of the present invention;

FIG. 5 is a rear view of a bowel packing device having a thermally-responsive section, in accordance with embodiments of the present invention;

FIG. 6 is a flowchart illustrating a method for use of a bowel packing device having a thermally-responsive section, in accordance with embodiments of the present invention;

FIG. 7 is a rear view of a bowel packing device having a thermally-responsive section, in accordance with embodiments of the present invention; and

FIG. 8 is a perspective view of a subject's abdominal cavity.

### DETAILED DESCRIPTION

Aspects of the present invention are generally directed to device for packing or retaining the bowels of a subject during a laparotomy or laproscopic surgical procedure. Such a device is referred to herein as a bowel packing device. The bowel packing device is configured to be operationally positioned within the subject to retain the bowels in a displaced position, and to provide a surgical space that allows a surgeon to perform the procedure. The bowel packing device comprises a support structure disposed therein that is configured to provide rigidity to portions of the device. As described in detail below, the support structure may be made from a number of different materials, such as malleable metal, carbon fiber composite, a thermally-responsive material, etc. Furthermore, in specific embodiments, the support structure comprises the substantial entirety of the device.

In embodiments in which carbon fiber or another fibrous material is used, the support structure is a group of such fibers formed into a body through a manufacturing process. As such, the use of carbon fiber does not include the use of a plurality of separate fibers dispersed through the material.

The use of a bowel packing device in accordance with embodiments of the present invention provides advantages over conventional sponges and towels not only in ease of use, but in improved patient outcomes. Specifically, the use of the bowel packing device provides for a reduction in adhesion formation (as has been demonstrated in rabbit adhesion trials) as a result of bowel packing as compared to bowel packing performed with sponges. Adhesions are due at least in part to fibers from sponges that remain in the abdominal cavity after the removal of sponges at the end of the surgery. As the bowel packing device of the present invention includes no exposed fibers, none can be left behind, eliminating at least one substantial cause of adhesions. The use of the bowel packing device also decreases bowel packing time, thereby decreasing the total surgical time. The overall surgical time reduction will depend on, in part, the number of times the bowel would need to be re-packed during the surgery. Therefore, in some embodiments the bowel packing devices allow for a reduction in operating room time, a reduction in anesthesia time, and a reduction in post-operative complications and morbidity associated with the use of surgical sponges used in current procedures.

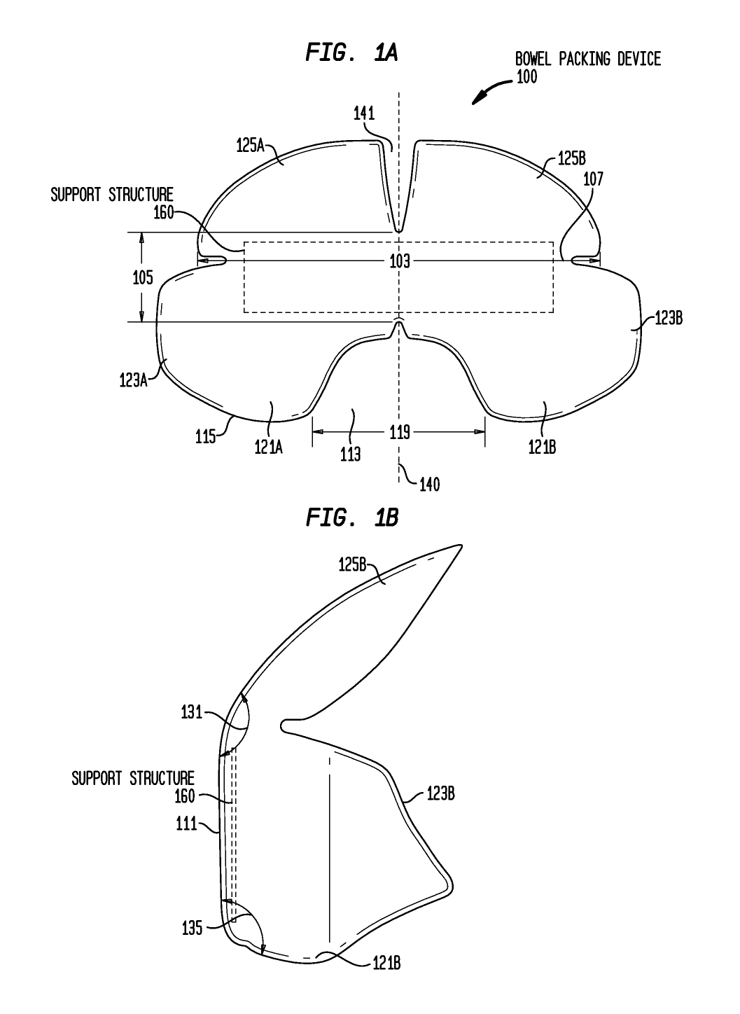

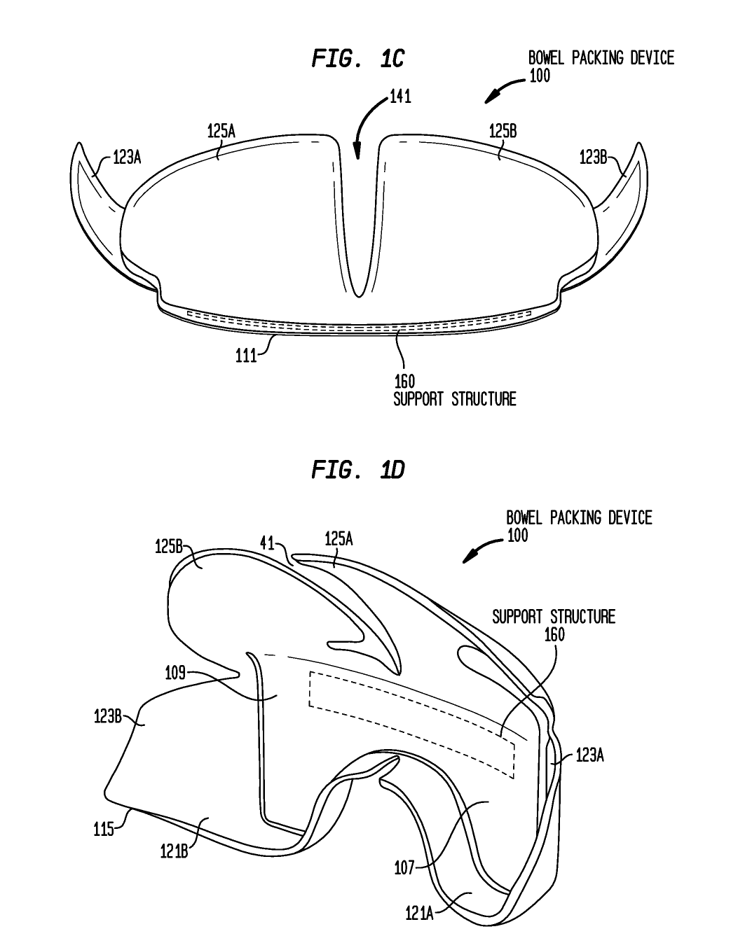

FIG. 1A is a front view of a bowel packing device 100 in accordance with embodiments of the present invention. As shown, bowel packing device 100 includes an essentially rectangular central portion 107 having a width 103 and a height 105. Device 100 further comprises first and second top flaps 125 separated by a notch 141, and first and second bottom flaps 121 separated by a notch 113. Additionally, body 107 further comprises two side flaps 123. Central portion 107 and flaps 121, 123, and 125, collectively form an essentially elliptical shape. More specifically, device 100 has an essentially elliptically-shape, that is generally symmetrical about a minor axis 140 of the device.

Notch 113 in FIG. 1A is a bell-shaped cut with in the bottom edge 115 of the device and is provided to accommodate the ventral medial part of the body in the sagittal plane, and designed to conform to, and provide space for, the spinal cord towards the ventral wall of the abdominal cavity).

In the embodiments of FIG. 1A, device 100 is formed from an elastomeric or polymeric compound, and device 100 further comprises a support structure 160 that is embedded in the device. More specifically, support structure 160 is a rectangular element that provides additional rigidity to central portion 107.

As previously noted, during a bowel packing procedure, a surgeon displaces the subject's bowels to create a space that allows the surgeon to perform the procedure. Device 100 is used to retain the bowels in this displaced position, thereby providing a barrier that maintains the surgical space. More specifically, a first surface 109 (FIG. 1D) of device 100 abuts the subject's bowel. In certain embodiments of the present invention, support structure 160 provides sufficiently rigidity to device 100 such that the device may retain the bowels in the displaced position without the need for additional surgical instructions. In such embodiments, device 100 is referred to herein as a self-retaining bowel packing device.

In other embodiments, surgical instruments, such as one or more retractor blades, are used to retain device 100 in its operable position. Specifically, the retractor blades interface with a second surface 111.

As used herein, bowels generically include bowel, intestine, and other abdominal organs that would need to be displaced in the abdominal cavity to allow for abdominal surgery. The standard retractor blade setup uses two blades that interface with the lateral sides of the body. Additionally, upon insertion of device 100 into a subject, side flaps 123 contact the lateral sides of the abdominal cavity and top flaps 125 contact the ventral side of the abdominal cavity. Side flaps 123 serve to aid in containing bowels that may protrude around the sides of the device in the abdominal cavity. The purpose of top flaps 123 is to help secure the bowels on the ventral side of the subject. In other words, device 100 is dimensioned to cover the bowels of the subject when operationally positioned within the abdomen of the subject.

Bowel packing device 100 is appropriately sized for bowel packing of a subject. That is, the device is dimensioned to allow for insertion into the abdominal cavity of the subject. For example, in adult humans, the size of the abdominal cavity is about 3.9 to 5.8 inches in the transverse plane at the height of the base of the ribs and about 7.6 to 11.3 inches in the coronal plane at the height of the base of the ribs. An appropriately sized device for bowel packing in a mammal having such dimensions is about 5.2 to about 7.5 inches overall height (from ventral to dorsal sides of the abdominal cavity upon placement) and about 8.7 to about 12.5 inches in overall width (from lateral side to lateral side of the abdominal cavity upon placement). However, it would be appreciated that device 100 may have different sizes and shapes, depending on, for example, the insertion technique, surgical procedure, subject, etc. In certain embodiments, portion 107 has a width 103 that is approximately 7.82 inches, and height 105 that is approximately 3.63 inches. In such embodiments, notch 112 has a height 117 of approximately 2.28 inches and a base width 119 of approximately 4.00 inches. Further details of the shape and of body of a bowel packing device in accordance with the embodiments of FIG. 1A are described in International Patent Application No. PCT/US2009/002495, filed on Apr. 22, 2009, the contents of which are hereby incorporated by reference herein. Specifically, bowel packing device 100 has substantially the same shape and size as provided for the bowel packing device described in PCT/US2009/002495.

It would be appreciated that the shape, size, location of notches, etc., of device 100 of FIG. 1A is merely illustrative, and different embodiments are within the scope of the present invention. For example, in certain embodiments, device 100 may having a thicknesses that varies throughout the device. In one specific such embodiment, the portion of device 100 at an intersection of the major axis and the minor axis is greater than the thickness of the body at the perimeter. The greater thickness of device 100 in central portion 107 for contacting the bowel provides greater rigidity, whereas the thinner, more flexible flaps allow for proper positioning of the barrier within the subject. In another embodiment, device 100 may include radial notches of essentially any shape that are independently selected. Notch shapes include, but fife not limited to V-shaped, U-shaped, and bell-shaped. Further details of variations for the shape, size, location of notches, etc., of device 100 may be as described in PCT/US2009/002495, the contents of which were previously incorporated by reference herein.

FIG. 1B is a side view of bowel packing device 100 of FIG. 1A. As shown in FIG. 1B, top flap 125 bend cephally at an angle 131 of about 140° to about 160° to the concave face 111 of the device. In the embodiment shown, the bottom edge of bottom flap 121 folds towards concave face 109 of the device at an angle 135 of about 95° to 115°.

FIG. 1C is a top view of bowel packing device 100 of FIG. 1A. As shown, top flaps 125 and side flaps 123 are angled towards the proximal face 109 of the device. FIG. 1D is a rear view of device 100 of FIG. 1A. As shown, top flaps 125, side flaps 123, and bottom flaps 121 are pointing cephally to define the concave proximal face 109 for contact with the bowel. In the embodiment shown, central portion 107 of device 100 is the thickest portion of the device, and has support structure 160 embedded therein that provides substantial rigidity in the portion for contact the bowel. As described in greater detail below, support structure 160 may a number of different arrangements, provide different degrees of rigidity to central region 107, and/or be positioned in different portions of device 100.

In the embodiments of FIGS. 1A-1E, device 100 is formed from an elastomeric or polymeric compound such as a silicone polymer, and support structure 160A is embedded therein. As used herein, “elastomeric compound” is understood as an elastic compound having an appropriate flexibility/rigidity, tear resistance, and sterilization resistance for use in the devices of the invention. Elastomeric compounds for use for manufacture of the device of the invention are sufficiently flexible to prevent damage from occurring to tissues or organs by contact with the device when in a non-compressed state. Elastomeric compound as used herein typically refers to an elastomeric polymer. The monomers that link to form the polymer are typically made from of carbon, hydrogen, oxygen and/or silicon. Examples of elastomeric polymers include Liquid Silicone Rubbers (LSR) and Silicone Encapsulants. In a specific embodiment of the invention, the elastomeric polymer is a “silicone polymer”. A “silicon polymer” is understood as any silicon-based polymeric material that has the appropriate flexibility/ rigidity, tear resistance, and sterilization resistance for use in the devices of the invention. In a further embodiment, the silicon polymer is optically clear. Elastomeric compounds for use in the device of the invention include, but are not limited to silicone, liquid silicone rubber (LSR), polydimethylsiloxane (PDMS), styrene butadiene rubber, styrene butadiene styrere (SBS) rubber, nitrile rubber, and polychloroprene (Neoprene). In one embodiment, silicon polymer is polydimethylsiloxane (PDMS) a silicon-based organic polymer. PDMS is optically clear, and is generally considered to be inert, non-toxic and non-flammable. In some embodiments, the material for the body is of sufficient flexibility to permit folding, compressing, or rolling of the device to allow for insertion through a retracted incision as small as 10 cm in diameter, while being of sufficient rigidity to expand after folding, compression, or rolling, and retain the, bowels for the duration of a surgical procedure when used in conjunction with retractor blades.

In an exemplary embodiment, the main body of the device includes an inner core of Sylgard® 184 (Dow Coming) polydimethylsiloxane polymer between 8 and 14 mm in thickness, to provide rigidity to the main body, encased in a layer of Sylgard® 186 to confer improved tear-resistance and durability to the barrier. Flaps are made of a tear resistant silicon polymer, with sufficient flexibility to allow for adjustment of the flaps in the abdominal cavity, while providing sufficient rigidity to retain the barrier in place. Exemplary peripheral flap materials include Sylgard® 186 between 2 and 8 mm in thickness, projecting from the main body at angles of between 20 and 60 degrees, and decreasing in thickness with distance from the main body.

When using more than one elastomeric compound for manufacture of the device, the compounds can be used together in any manner. For example, a polymer with the desired rigidity can be coated with a polymer having greater smoothness. The body can be composed of one polymer, and the flaps can be composed of one or more other polymers to provide varying amounts of rigidity to the central portion and the flaps.

Further, in an embodiment, at least some portions of the device are made of a clear material which allows the bowels to be visually monitored throughout the procedure, an advantage not allowed by the sponges used in current procedures. Further, the use of an elastomeric material provides for retention of both moisture and warmth in the abdominal cavity as compared to packing methods using surgical sponges.

Embodiments of the barrier (including the collapsible barrier) may be made, at least in part, from thermoplastic elastomers, such as by way of example, styrenic block copolymers, polyolefin blends, elastomeric alloys, TPU, thermoplastic coployester, and thermoplastic polyamides, polysulfide rubber, and/or thermoplastic vulcanizates. Still further, thermoset elastomers, including polyisoprene, may be used to make at least some portions of the barrier. Saturated rubbers may also be used, such as, for example, EPM and EPDM, Epichlorohydrin rubber, polyacrylic rubber, florosilicone rubber, fluoroelastomers, perfluoroelastomers, polyether bock amides, cholosulfonated polyethylene, ethylene-vinycl acetate. Non-elastomeric polymers may also be used to make the barrier, including PTFE, PU, PTE, LDPE, Cross-linked PE, HDPE, PE, Polypropylene, PEEK, PVC, polycarbonate, Polystyrene, and/or PEI. Composite materials may also be used, which may include the above-mentioned polymers and materials combined with reinforcing fibers, fillers, woven materials, polymer foam inserts, etc.

Polymers with relatively low Tg/softening points that would deform with steam sterilization may be used to manufacture the collapsible barrier. An embodiment of the present invention includes features/the use of materials that reduce the likelihood that the barrier may be reused, thus reducing the spread of disease and post-operative complications.

As previously noted, in certain embodiments body 102 is formed from a material having a desired level of tear resistance. Tear resistance is the resistance of a material to initial tearing while tear strength represents the force required to tear a pre-slit material. For use in some embodiments, an un-slit, material needs to have no visible tears develop upon application of 100 of shear force. The amount of shear force required to tear pre-slit material may also be determined to identify potential failure modes of the barrier. In order to determine if Sylgard® 184 and or Sylgard® 186 may be able to withstand expected shear forces applied by the retractor blades on the body of the barrier, both tear resistance and tear strength of the material may be determined. Sylgard® 184 and 186 may be compared to each other to determine the most tear-resistant material. Force thresholds may be determined from measurements made in a simulated abdominal cavity.

As noted above, embedded in device 100 is a support structure 160 that may also be made from a number of different materials having different properties. For example, in certain embodiments, support structure 160 is made in whole or in part from a malleable metal, including, but not limited to, stainless steel and/or aluminum. The use of the malleable metal allows the surgeon to bend or conform the support structure during the bowel packing procedure. In other embodiments, any metal, regardless of whether it is malleable, may be used for the whole, or a portion of, support structure 160. By way of example, in one such embodiment, support member 160 comprises a titanium member. Also, in alternative embodiments, support structure 160 is made in whole or in part from a carbon fiber composite structure and/or graphite epoxy. In yet other alternative embodiments, the reinforcement structure is made in pail, entirely or substantially entirely of Kevlar, fiberglass and/or a cellulose fiber.

In still further embodiments, support structure 160 is made from a substantially rigid elastomeric compound. In such embodiments, support structure 160 has a rigidity that exceeds the rigidity of the remainder of device 100.

Support structure 160 may be a monolithic structure or a composite structure. As such, support structure 160 may have the same material throughout, or include a plurality of different materials. As noted above, in certain embodiments, support structure 160 is made from a conformable material (malleable metal, certain polymers, etc.). In embodiments in which such elements are used in a composite structure of different materials, certain sections of the structure may be more bendable or conformable than others.

In still other embodiments, support structure 160 may be made from a material that is biased or has a propensity to bend in a certain direction. For example, in certain such embodiments, support structure 160 may be biased such that, when inserted into the subject, the structure exerts a force in the direction of the subject's bowels.

As detailed further below, support structures in accordance with embodiments of the present invention may also have different shapes and sizes, or be positioned at different locations within the body of the bowel packing device.

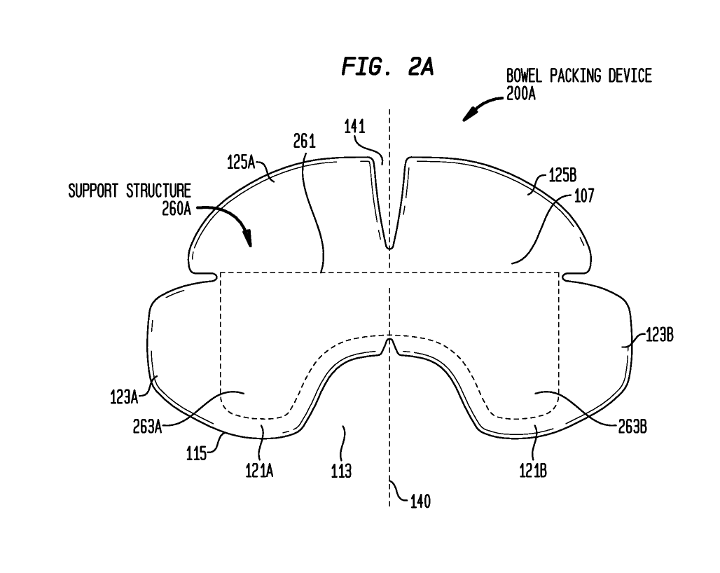

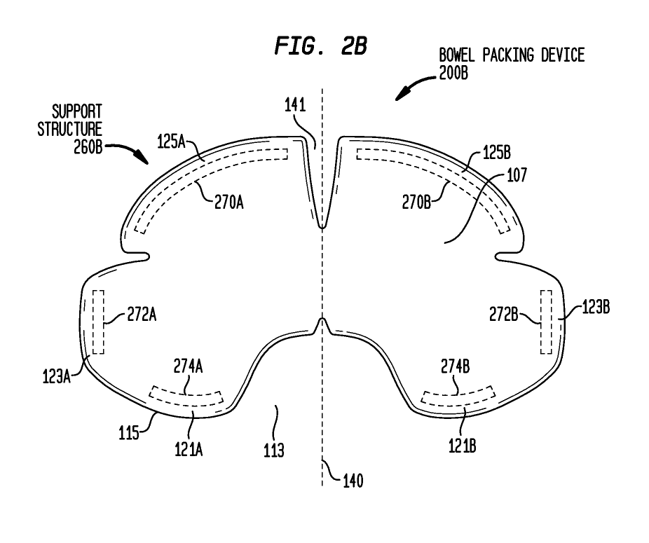

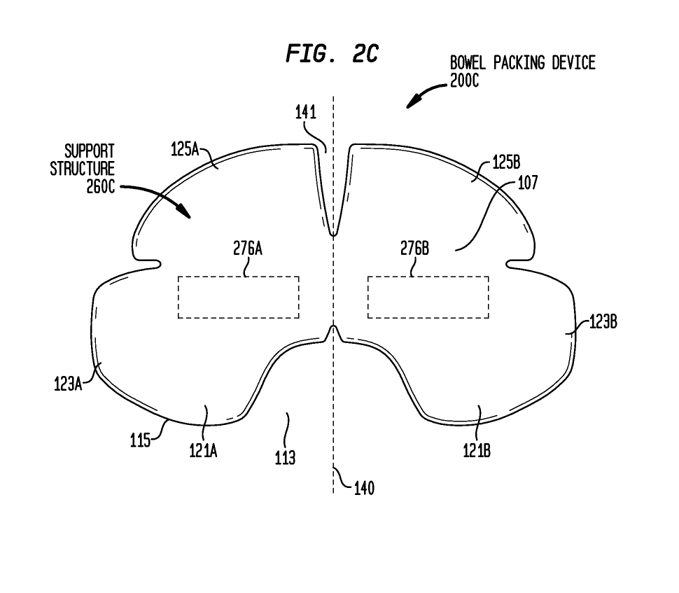

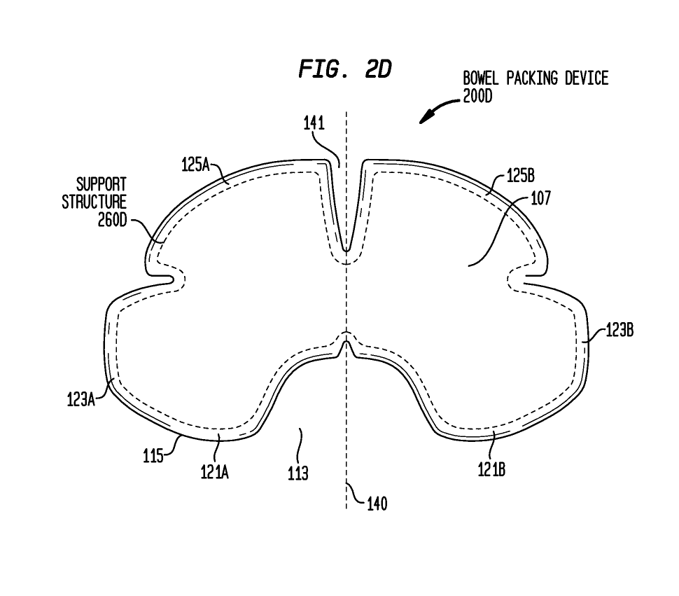

FIGS. 2A-2D are front views of bowel packing devices having different support structures in accordance with embodiments of the present invention. FIG. 2A is a front view of one such bowel packing device 200A.

Device 200A is similar to device 100 of FIGS. 1A-1E, and includes a central portion 107 and flaps 121, 123 and 125. Bowel packing device 200A further comprises a planar support structure 260A embedded in the device. In this embodiment, support structure 260A includes a rectangular shape region 261 disposed in central portion 107, and two projections 263 that extend from central region 107 into flaps 121. In the illustrative embodiment of FIG. 2A, projections 263 have a area there between that is generally the same shape as notch 113.

It would be appreciated that the shapes and locations for region 261 and projections 263 provided above are merely illustrative and do not limit embodiments of the present invention. For example, in other embodiments, projections 263 may be rectangular elements that extend from region 261. In still other embodiments, region 261 may have a circular, oval or other shapes. In further embodiments, projections 263 may also, or instead, extend into flaps 123 and/or 125.

Similar to the embodiments of FIGS. 1A-1E, in the embodiments of FIG. 2A, device 200A is an elastomeric compound, and support structure 2.60A is formed from a material that has a greater rigidity than the elastomeric compound. Support structure 260A may be formed from any of the materials described above with reference to FIGS. 1A-1E.

FIG. 2B is a front view of another bowel packing device 200B in accordance with embodiments of the present invention. Device 200B is similar to device 100 of FIGS. 1A-1E, and includes a central portion 107 and flaps 121, 123 and 125. Bowel packing device 200B further comprises a support structure 260B embedded in the device. In this embodiment, support structure 260B comprises a plurality of linear members 270, 272, and 273 embedded in the flaps of body 102. More specifically, members 270A, 270B are embedded near the outer edges of flaps 125A, 125B, respectively. Additionally, members 272A, 272B are embedded near the outer edges of flaps 123A, 123B, respectively, while members 274A, 274B are embedded near the outer edges of flaps 121A, 121B, respectively.

In this embodiment, members 270 are generally planar, but have a curved shape that follows the curve of the edge of flaps 125. Members 274 also have a curved shape that that follows the curve of the edge of flaps 121, but members 123 are substantially straight.

It would be appreciated that the shapes and locations for members 270, and 274 provided above are merely illustrative and do not limit embodiments of the present invention. For example, in other embodiments, any of the members 270, 272 and 274 nay be omitted from the device.

Similar to the embodiments of FIGS. 1A-1E, in the embodiments of FIG. 2B, device 200B is an elastomeric compound, and members 270, 272 and 274 are formed from a material that has greater rigidity than the elastomeric compound. Members 270, 272 and 274 may be formed from any of the materials described above with reference to FIGS. 1A-1E.

FIG. 2C is a front view of another bowel packing device 200C in accordance with embodiments of the present invention. Device 200C is similar to device 100 of FIGS. 1A-1E, and includes a central portion 107 and flaps 121, 123 and 125. Bowel packing device 200C further comprises a support structure 260C embedded in the device. In this embodiment, support structure 260C comprises two planar rectangular members 276 embedded in central portion 107 on opposing sides of axis 140. It would be appreciated that the shapes and locations for members 276 are merely illustrative and do not limit embodiments of the present invention.

Similar to the embodiments of FIGS. 1A-1E, in the embodiments of FIG. 2C, device 200C is an elastomeric compound, and members 276 are formed from a material that has greater rigidity than the elastomeric compound. Members 276 may be formed from any of the materials described above with reference to FIGS. 1A-1E.

FIG. 2D is front view of another bowel packing device 200D in accordance with embodiments of the present invention in which support structure 260D comprises the substantial entirely of device 200D. More specifically, support structure 260D has and essentially elliptical shape that is generally symmetrical about a minor axis, and is dimensioned to cover the bowels of the subject when operationally positioned within the abdomen of the subject. In the embodiments of FIG. 2D, support structure 260D is similar to device 100 of FIGS. 1A-1E, and includes a central portion 107 and flaps 121, 123 and 125. Device 200D also includes a layer of elastomeric compound disposed around support structure 260D. Support structure 260D may be formed form any of the materials described above with reference to FIGS. 1A-1E.

As noted above, FIG. 2D illustrates embodiments of the present invention in which support structure 260D is embedded in a elastomeric compound. In other embodiments of the present invention, the elastomeric compound is not used and support structure 260D and body 102 are the same element. That is, in such embodiments, device 200D is formed from any of the materials described above with reference to FIGS. 1A-1E.

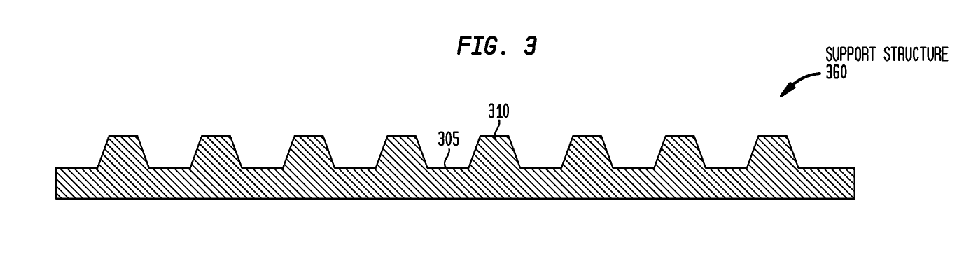

The above embodiments of the present invention have been generally described with reference to support structures in the form of planar elements having a substantially consistent thickness. FIG. 3 is a cross-sectional view of a support structure 360 in accordance with alternative embodiments of the present invention in which the support structure has varying thickness. Specifically, support structure includes protrusions 310 separated by valleys 305 as shown. In specific embodiments, protrusions 310 may be foamed in strips that are parallel to one another. In another embodiment, protrusions 310 may be in the form of checkered protrusions, discrete unconnected protrusions, or a waffle pattern.

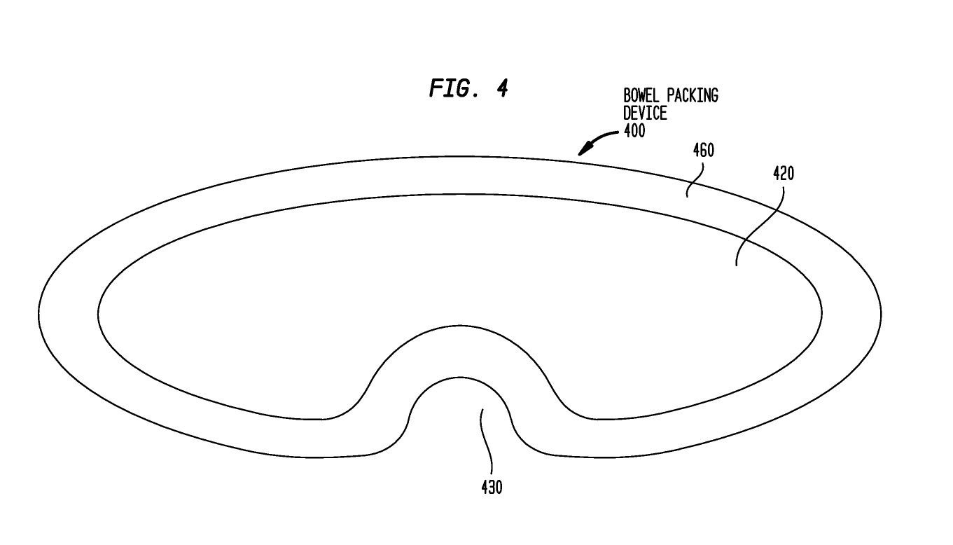

FIG. 4 is a perspective view of another bowel packing device 400 in accordance with embodiments of the present invention. As shown, device 400 includes a support structure 460 in the form of an outer frame that surrounds an interior diaphragm 420. Diaphragm 420 is formed from an elastomeric compound, and may be, in certain embodiments, a plastic film having substantially uniform thickness.

As shown, support structure 460 has an essentially elliptical shape, and includes a notch 430 therein. Notch 430 is sized, shaped and located to accommodate a subject's spine when device 400 is inserted, into the subject.

Additionally, support structure 460 is formed form any of the materials described above with reference to FIGS. 1A-1E. Support structure 460 is, in certain embodiments, embedded in an elastomeric compound as described elsewhere herein.

FIGS. 1A-4 generally illustrate embodiments of the present invention in which a support structure is embedded in an elastomeric material. In other embodiments of the present invention, the device is substantially or entirely formed from the support structure, and no elastomeric coating or body is used. In such embodiments, the support structure is formed from a material that is biocompatible and will not damage the subject's tissue.

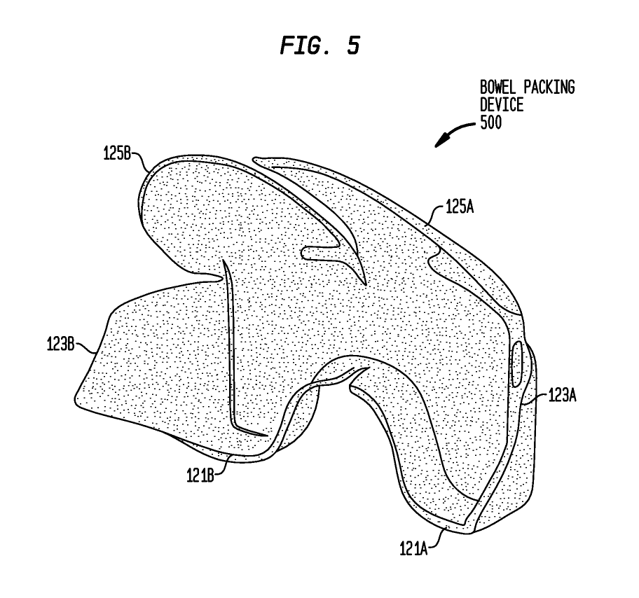

The above embodiments of the present invention are generally directed to variations of a bowel packing device that includes a support structure in the form one or more rigid members. FIGS. 5-7 illustrate embodiments of the present invention in which the support structure is in the form of thermally-responsive materials that form the whole or portion of the device.

Specifically, FIG. 5 is a review view of a bowel packing device 500. Device 500 is similar to device 100 of FIGS. 1A-1E, and includes a central portion 107 and flaps 121, 123 and 125. However, in contrast to the above embodiments of the present invention in which the devices are formed from a thermally-stable material, in the embodiments of FIG. 5, device 500 is formed from a thermally-responsive material. As used herein, a thermally-stable material is a material that has specific material properties at or below a subject's body temperature, and that maintains those specific to temperatures significantly above the subject's body temperature. Also as used herein, significantly above a subject's body temperature is a temperature that a device at or near this temperature could damage the subject's tissue if inserted into the subject. In one specific example, such a temperature is 10 degrees Fahrenheit or more above the subject's body temperature.

A thermally-responsive material is defined herein as a material that has specific material properties, particularly stiffness, at or near a subject's body temperature, but has different material properties, particularly decreases stiffness, at temperatures slightly above the subject's body temperature. Also as used herein, slightly above a subject's body temperature is a temperature that a device at or near this temperature would not damage the subject's tissue if inserted into the subject. In one specific example, such a temperature is 5-15 degrees Fahrenheit above the subject's body temperature.

Examples of thermally-responsive materials that may he used in embodiments of the present invention include, but are not limited to, low softening point foamed. Thermoplastics, low softening point thermoplastics, low density foamed Ethylene Vinyl Acetate (EVA), foamed polyethylene, foamed polyurethane, foamed polyester . . .

As such, in the embodiments of FIG. 5, prior to insertion of device 500 into a subject, the devise is heated to a temperature such that device 500 becomes conformable such that it can be inserted and properly positioned abutting the bowels. As device 500 cools from this temperature to be approximately at the subject's body temperature, the device becomes more rigid and essentially locks into it's operable, bowel retaining position.

As previously noted, different thermally-responsive materials may be used in embodiments of the present invention and the temperature required to make the device sufficiently conformable will vary depending on the thermally-responsive material. In embodiments of the present invention, the temperature required for conformability is above the subject's body temperature, but is low enough that, device 500 can be safely handled by the surgeon and such that, when device 500 is inserted, the temperature will not injure the subject.

FIG. 5 illustrates embodiments of the present invention in which the device is entirely formed from a thermally-responsive material. In alternative embodiments of the present invention, only one or more sections of the device is formed from a thermally-responsive material. In such embodiments, the thermally-responsive material may be located in areas in which added rigidity is desired during operation, such as the locations shown for support structures in the embodiments of FIGS. 1A-1E, 2A-2D, and FIG. 3. That is, the support members described above in these embodiments could be replaced with thermally-responsive materials.

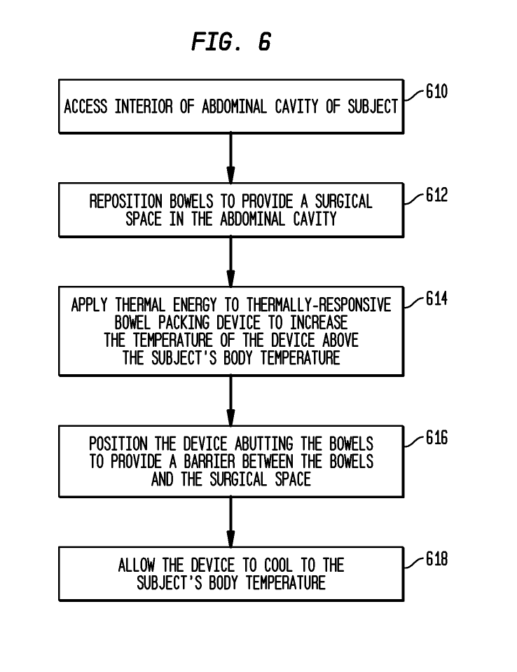

FIG. 6 is a flowchart illustrating an exemplary method 600 for using a bowel packing device having a section formed from a thermally-responsive material. Method 600 begins at step 610 where a surgeon accesses the interior of an abdominal cavity of the subject. At step 612, the surgeon repositions the bowels of the subject to provide a surgical space in the abdominal cavity.

Next, at step 614, thermal energy is applied to the device to increase the temperature of the device, thereby change the stiffness of the thermally-responsive material in the device. At step 616, the device is positioned in a manner such that it abuts the bowels and provides a barrier between the bowels and the surgical space. More specifically, at least a portion of the device is conformed to the general profile of the bowels and/or the abdominal cavity by plastically deforming the bowel barrier while the portion is above the subject's body temperature. At step 618, the device is allowed to cool to the subject's body temperature such that the device obtains sufficient rigidity to retain the bowels. Moreover, in some embodiments, the lowering of the temperature “locks” the device in the new configuration. Insertion and positioning of the device into the subject may be done by hand, in the case of a laparotomy, or remotely using a probe or the like in the case of a laparoscopic procedure.

As noted above, thermal energy is applied to a device having a thermally-responsive section to increase the temperature, and temporality decrease the rigidity of the thermally-responsive section. Thermal energy may he applied to the device using a number of different methods. In one embodiment, the thermal energy is applied using thermal radiation from, for example, a heat lamp, or the like, or placing the device in an autoclave set at a relatively low temperature or limiting the temporal exposure of the barrier placed in an autoclave at a relatively high temperature. In other embodiments, the thermal energy is applied through the use of convention and/or conduction heat transfer. For example, the device might be placed in a warm-water or warm fluid bath, the device may be placed in an oven, an element emitting relatively high amounts of thermal energy may be placed against the barrier, etc. In still other embodiments, the barrier may be rubbed to introduce thermal energy via friction, or the material may be a material that increases its temperature through repeated bending, flexing, compression, expansion, etc. In further embodiments, the thermal energy may be applied through deliver of an electrical current or upon exposure to non-thermal radiation of a given frequency (e.g., light). In some embodiments, the thermal energy is applied via microwaves of a microwave oven. In one such exemplary embodiment, the device includes a water-filled reservoir adjacent the thermally-responsive material. Upon exposure of the water to microwaves, the temperature of the water increases, thereby heating the thermally-responsive material. These different methods of application of thermal energy are merely illustrative, and any means that will permit transfer of thermal energy to the device to decrease the stiffness of the thermally-responsive material may be used in embodiments of the present invention.

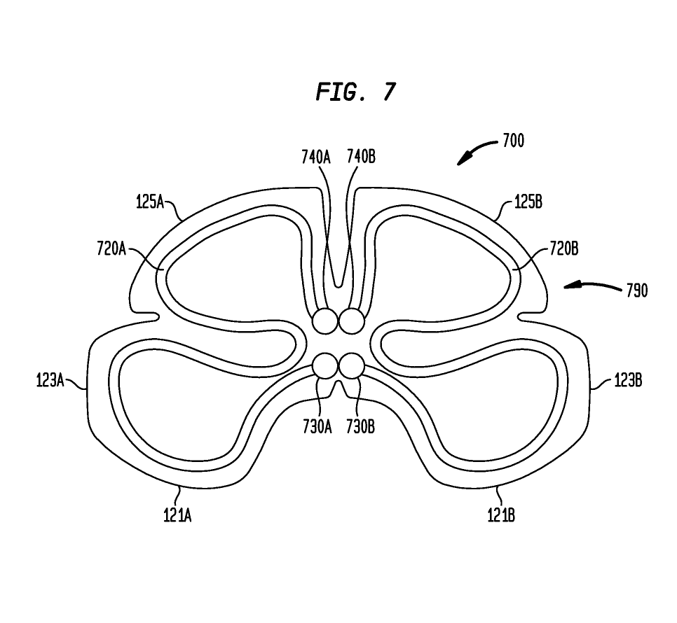

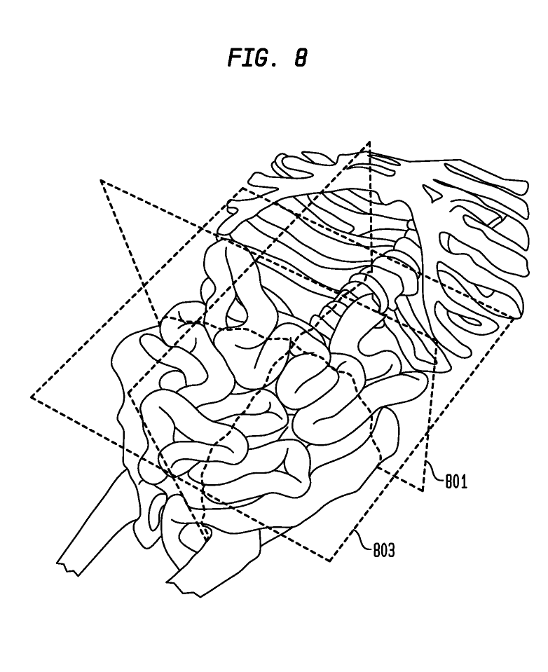

FIG. 7 is a front view of a bowel packing device 700 formed from a thermally-responsive material as described above. In this illustrative embodiment, device 700 includes a heating system 790 for application of thermal energy to the thermally-responsive material.

As shown, device 700 is substantially formed from a thermally-responsive material. Similar to the embodiments described above, device 700 includes a central portion 107, and flaps 121, 123 and 125. Heating system 790 includes a fluid circuit embedded in, or disposed on, the device. The circuit comprises an inlet port 730A coupled to an outlet port 740A via conduit 720A. Circuit also comprises an inlet port 730B coupled to an outlet port 740B via conduit 720B. To increase the temperature of the thermally-responsive material, a heated fluid is directed into inlet ports 730 where it then travels to fluid outlets 740. Due to the presence of the heated fluid in the fluid passages 720, the thermally-responsive material is heated to an appropriate temperature sufficient to increase the conformability to enable insertion into the subject. Specifically, once the thermally-responsive material is sufficiently heated, the circulation of the heated fluid is ceased, and device 700 is inserted into the subject.

FIG. 7 illustrates embodiments including two inlet ports 730 and two outlet ports 740. In other embodiments, the device, includes one inlet port and one, outlet port. Ports 730, 740 and conduits 720 may be embedded within the device, or positioned one or more surfaces of the device.

In specific embodiments of FIG. 7, conduits 720 are collapsible. When heating of the material is required, conduits 720 expand due to the flow of fluid there through, and device expands as a result. That is, the introduction of fluid into conduits 720 expands the collapsible barrier, expanding it to dimensions sufficient to retain interior organs/bowels. In an exemplary embodiment, the collapsed device is inserted into the abdominal cavity via a cannula of a trocar, and a flexible tube extends from the collapsible device through the cannula and out of the subject to a fluid pump. The fluid pump is activated by a surgeon or other type of technician, and the fluid pump pumps fluid through the flexible tube and into conduits, thereby causing the collapsible device to expand.

In an alternate embodiment, instead of or in addition to the fluid circuit of FIG. 7, an electrical circuit may be used to heat and/or cool the thermally-responsive material. Such a circuit may utilize electrical resistance to heat the material and/or utilize a Peltier circuit to cool the material.

In some embodiments, instead of adding thermal energy to the thermally-responsive material to raise the temperature above that of the subject, thereby decreasing the stiffness of the material, thermal energy is removed from the material to lower the temperature of the material. By way of example, the material may have a suitable flexibility at standard room temperature to conform to the bowels, and may become suitably stiff when cooled below room temperature. In such embodiments, the thermally-responsive material may be thermally insulated from the recipient such that the relatively lower temperature of the material does not adversely affect the subject.

As previously noted, bowel packing devices in accordance with embodiments of the present invention are preferably made in different sizes for use in subjects of different sizes (e.g., children and adults). FIG. 8 illustrates relative dimensions of the average human abdomen that were used to determine the dimensions of an exemplary device for use in adult human with the transverse 801 and coronal 03 planes indicated. Using the measurements of the adult human abdominal cavity and the devices, the appropriate dimensions for a bowel packing device can be determined for use in a subject other than an adult human provided with the dimensions of the abdominal cavity (human child, dog, cat, other mammal). Anthropologic data may be used to determine the small, medium, and large sizes designed to fit at least 95% of the adult human population. This flexibility of the device allows it to conform to cavities that may otherwise be too big or too small. In a embodiment, the small size will be about 5.20 inches total in height and about 8.70 inches total in width; the large size will be about 7.50 inches in height and about 12.50 inches in length; the medium size of the device is about 6.53 inches in total height, including the body and flaps, and about 10.92 inches wide.

The packing devices of the invention can also include other components such as coatings to reduce sticking of the device to the bowel by coating with polymers, particularly biocompatible polymers, of with commercially available coatings such as Seprafilm®. The coatings may be drug eluting. The coatings may be applied by bulk application, molecular conjugation with the body material, or through nanostructure formation. Examples of possible coatings include: SEPRAFILM®, INTERCEED®, SIROLIMUS®, PACLITAXEL®, EVEROLIMUS®, TRANILAST®, DACRON®, SPRAYGEL®, ADHffiIT®, TEFLON®, PRECLUDE® Gore, SyntheMed REPEL-CV®, DuraGen, ADCON'M P (Gliatech), REPEUM and RESOLVE™ (Life Medical Sciences), INTERGEL™ (formerly LUBRICOAT®), icodextrin, hyaluronic acid, heparin, dextran, tissue plasminogen activator, corticosteroids, non-steroid inflammatory drugs (NSAIDS) such as ibuprophen, chondroitin sulfate, carboxymethylcellulose, dexamethosane, tissue plasminogen including recombinant tissue plasminogen, oxyphenbutazone, collagen, collagen inhibitors, polylactic acid, polyglycolic acid, alginic acid, polycaprolactone, glycosaminoglycans, polyethylene oxide (PEO), polyethylene oxidepolypropylene oxide copolymer in any monomeric ratio (PEG-PPO-PEG), hydroxy ethyl methyl acrylate (HEMA), poly(N-isopropylacrylamide) (NIPAAm), polytetraflouroethylene (PTFE), polyesters, and silane, or modification by radio frequency gas discharge (RFGD), and radiation grafting. polytetrafluoroethylene (PTFE), polylactic acid, polyglycolic acid, alginic acid, polycaprolactone, glycosaminoglycans, HEMA, ePTFE, polyesters, carboxymethylcellulose, dexamethasone, tissue plasminogen including recombinant tissue plasminogen, oxyphenbutazone, corticosteriods, icodextrin, hyaluronic acid, hyaluronan, and collagen inhibitors.

Alternatively, packing devices can be coated with agents, for example, anti-microbial agents such as anti-viral agents or anti-bacterial agents. The use of such agents may be useful for the protection of the subject as well as the surgical staff and to reduce the possibility of transmission of infection from subjects infected with HIV, hepatitis, especially drug-resistant forms of hepatitis, methicillin resistant staphylococcus aureus (MERSA), etc.

Embodiments of the present invention have been primarily described with reference to support structures embedded or disposed in the device. However, as noted above, in certain embodiments, the support structure may comprise the substantial entirety of the device and, as such, the device is formed from the support structure. Additionally, in other embodiments, the support structure is not necessarily disposed or embedded in another device, but rather may be, in certain embodiments, disposed on the surface of the device.

Also as noted above, bowel packing devices in accordance with embodiments of the present invention may be inserted into a subject via a laparotomy, or via a laproscopic procedure. In embodiments in which the device is configured for insertion via a laproscopic procedure, the device is sufficiently collapsible that the barrier may be inserted into an abdomen via a trocar, gel port or substantially small incision, the size of which is known in the art. The size of a such an incision is small when compared to the incision typically made through the ventral side of the subject during a laparotomy.

The collapsible device may be collapsed (e.g., rolled, folded or otherwise bunched together) to fit into the cannula of the trocar, etc. Sufficient three applied to the collapsible device causes the device to move through the cannula of the trocar and into the abdominal cavity. Once in the abdominal cavity, the device is uncollapsed or expanded (e.g., unrolled, unfolded, unbunchned, etc.) to expand to the configuration(s) detailed herein. The device has sufficient structural rigidity after it is expanded such that it maintains the bowels in a retained state. In such embodiments, the support structure is positioned within the body, and or is made from a sufficiently conformable material, so as to facilitate the required collapsing and expansion.

Once the device is no longer needed in the abdomen, the device may be re-collapsed so that it may be withdrawn from the abdomen through the cannula of the trocar and/or through the in in the abdomen.

The invention described and claimed herein is not to be limited in scope by the specific embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All documents, patents, journal articles and other materials cited in the present application are hereby incorporated by reference.

Reference herein to “one embodiment” or “an embodiment” means that a particular feature, structure, operation, or other characteristic described in connection with the embodiment may be included in at least one implementation of the invention. However, the appearance of the phrase “in one embodiment” or “in an embodiment” in various places in the specification does not necessarily refer to the same embodiment. It is further envisioned that a skilled person could use any or all of the above embodiments in any compatible combination or permutation.

What is claimed is:

1. An elastomeric device for packing the bowels of a subject comprising:
a central portion and one or more flaps collectively manually positionable within the subject to retain the bowels of the subject in an operational, displaced position and to provide a surgical operational space; and
a support structure disposed in at least one of the central portion and the flaps configured to provide rigidity to the device.

2. The device of claim 1, wherein the central portion and the one or more flaps form an essentially elliptical shape that is generally symmetrical about a minor axis of the device.

3. The device of claim 1, wherein the support structure is one or more malleable metal members.

4. The device of claim 1, wherein the support structure is one or more stainless steel members.

5. The device of claim 1, wherein the support structure is one or more aluminum members.

6. The device of claim 1, wherein the support structure is one or more carbon fiber composite bodies.

7. The device of claim 1, wherein the support structure is a graphite epoxy composite structure.

8. The device of claim 1, wherein the support structure is a Kevlar member.

9. The device of claim 1, wherein the support structure is one or more fiberglass bodies.

10. The device of claim 1, wherein the support structure is a cellulose fiber member.

11. The device of claim 1, further comprising:
a cut-out located on the minor axis of the device, wherein the cut-out is dimensioned to accommodate the spine of the subject.

12. The device of claim 11, wherein a portion of the support structure is disposed in at least one of the flaps.

13. The device of claim 1, wherein the body is sufficiently flexible to bend around the spine of the subject during packing of the bowels.

14. The device of claim 1, wherein the support structure is a malleable metal, and wherein the body is substantially formed of the malleable metal.

15. A device for packing the bowels of a subject comprising:
a central portion and one or more flaps collectively manually positionable within the subject to retain the bowels of the subject in an operational, displaced position and to provide a surgical operational space,
wherein at least one of the central portion and the one or more flaps body has a section formed from a thermally-responsive material having a first stiffness when the material is at a first temperature that is above the subject's body temperature, and a second, greater stiffness when the body temperature is approximately at the subject's body temperature.

16. The device of claim 15, wherein the device is substantially is formed of the thermally-responsive material.

17. The device of claim 15, further comprising a heat transfer device configured to transfer thermal energy to and/or from the thermally-responsive material.

18. The device of claim 17, wherein the heat transfer device comprises at least one of an electrical circuit and a fluid circuit proximate the thermally-responsive material.

19. A method of packing bowels of a subject with a device including a central portion and one or more flaps, wherein the device has a section formed from of a thermally-responsive material having a first stiffness when the material is at a first temperature that is above the subject's body temperature, and a second, greater stiffness when the body temperature is approximately at the subject's body temperature, comprising:
accessing an interior of an abdominal cavity of the subject;
repositioning the bowels to provide a surgical space in the abdominal cavity;
adding thermal energy to the device to increase the temperature of the device;
positioning the device abutting the bowels; and
allowing the device to cool to the subject's body temperature such that the device provides a barrier between the bowels and the surgical space.

20. The method of claim 19, wherein positioning the device abutting the bowels, comprises:
conforming a section of the device to the general profile of the bowels, and
placing the section in contact with the bowels while the device is above the subject's body temperature.

21. The method of claim 19, wherein allowing the thermally-responsive material to cool to the subject's body temperature such that the device provides a barrier between the bowels and the surgical space, further comprises:
allowing the thermally-responsive material to cool so as to provide sufficient rigidity to retain the bowels without the use of an additional surgical instrument.

22. The method of claim 19, wherein the body further comprises a heat transfer device, and wherein the method comprises:
transferring, with the heat transfer device, thermal energy to and/or from the thermally-responsive material.

23. The method of claim 22, wherein the heat transfer device comprises at least one of an electrical circuit and a fluid circuit proximate the thermally-responsive material.
